(19) United States
(12) Patent Application Publication
(10) Pub. No.: US 2024/0213535 A1
(43) Pub. Date: Jun. 27, 2024

(54) ELECTROLYTE SOLUTION FOR RECHARGEABLE LITHIUM BATTERY AND RECHARGEABLE LITHIUM BATTERY INCLUDING THE SAME

(71) Applicant: SAMSUNG SDI CO., LTD., Yongin-si (KR)

(72) Inventors: Harim LEE, Yongin-si (KR); Tae Hyon BAE, Yongin-si (KR); Sanghyung KIM, Yongin-si (KR); Seunghyeon SON, Yongin-si (KR); Yunhee KIM, Yongin-si (KR); Arum YU, Yongin-si (KR)

(21) Appl. No.: 18/489,734

(22) Filed: Oct. 18, 2023

(30) Foreign Application Priority Data
Dec. 15, 2022 (KR) 10-2022-0176213

(51) Int. Cl. H01M 10/0567; H01M 10/0525

(52) U.S. Cl. CPC H01M 10/0567; H01M 10/0525

(57) ABSTRACT

An electrolyte solution for a rechargeable lithium battery includes a non-aqueous organic solvent, a lithium salt, and an additive, where the additive includes a first compound that is CsPF₅ or a compound represented by Chemical Formula 1, a second compound represented by Chemical Formula 2, and a silver salt. In addition, a rechargeable lithium battery including the electrolyte solution is also provided.

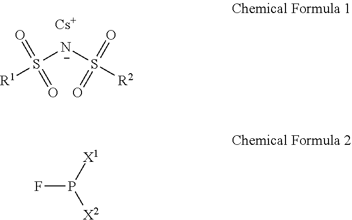

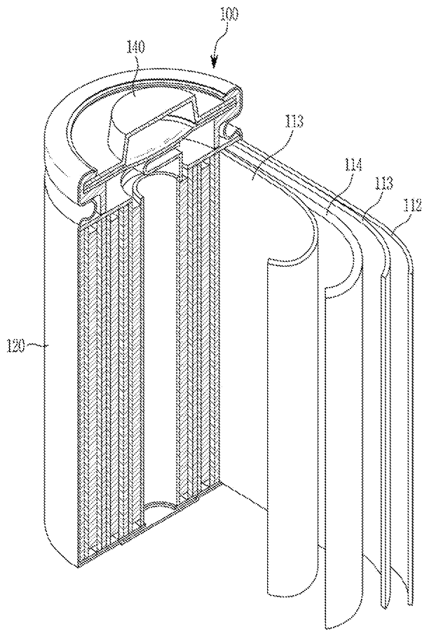

### CROSS-REFERENCE TO RELATED APPLICATION

This application claims priority to and the benefit of Korean Patent Application No. 10-2022-0176213 filed in the Korean Intellectual Property Office on Dec. 15, 2022, the entire content of which is incorporated herein by reference.

### BACKGROUND

### 1. Field

One or more embodiments of the present disclosure relate to an electrolyte solution for a rechargeable lithium battery and a rechargeable lithium battery including the electrolyte solution.

### 2. Description of the Related Art

A rechargeable lithium battery is widely utilized as a driving power source for mobile information terminals such as smart phones and laptops because it is easy to carry while implementing high energy density. Recently, a rechargeable lithium battery having (e.g., secured with) relatively high capacity, high energy density, and high safety has been actively studied for utilization as a power source for driving a hybrid vehicle or an electric vehicle, or as a power source for power storage.

In a rechargeable lithium battery, an electrolyte plays an important role in delivering and transporting lithium ions. An electrolyte solution including an organic solvent and a lithium salt is typically (e.g., most commonly) utilized because it can exhibit extremely high ionic conductivity. In addition, the electrolyte solution also plays an important role in determining safety and performance of the rechargeable lithium battery.

Recently, as a high-capacity, high-energy density battery is required or desired, it is necessary or desired to design a battery drivable at a high voltage of about 4.5 V or higher, while an electrode is highly densified. However, under harsh operation conditions such as high voltage, the positive electrode is deteriorated, and lithium dendrite grows on the surface of the negative electrode, which may accelerate side reactions between the electrodes and the electrolyte solution, and thus deteriorate battery cycle-life (and/or life-cycle) and cause a battery safety issue due to gas generation and/or the like.

In order to address the battery safety issue, methods to suppress or reduce the side reactions with the electrolyte solution by surface-treating the electrodes for protection have been proposed. However, the surface treatment of the positive electrode may provide insufficient protection effect during the high voltage driving conditions, and the surface treatment of the negative electrode has been reported to have a problem of deteriorating capacity. Accordingly, in the design of high-capacity electrodes capable of driving at a high voltage, development of an electrolyte solution improving safety and performance of batteries is still desired or required.

### SUMMARY

One or more aspects of embodiments of the present disclosure are directed toward an electrolyte solution for a rechargeable lithium battery with stable film formation, improved rapid charging characteristics, suppressed increase in internal resistance, secured battery safety and high-temperature reliability under high-voltage driving conditions, and improved capacity characteristics and cycle-life characteristics, and a rechargeable lithium battery including the same.

Additional aspects will be set forth in part in the description, which follows and, in part, will be apparent from the description, or may be learned by practice of the presented embodiments of the present disclosure.

In one or more embodiments of the present disclosure, an electrolyte solution for a rechargeable lithium battery includes a non-aqueous organic solvent, a lithium salt, and an additive, wherein the additive includes a first compound (that is CsPF₆, a compound represented by Chemical Formula 1, or a combination thereof), a second compound represented by Chemical Formula 2, and a silver (Ag) salt.

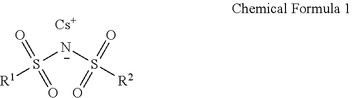

In Chemical Formula 1, R¹ and R² may each independently be a fluoro group or a C1 to C4 fluoroalkyl group substituted with at least one fluoro group.

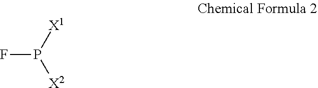

In Chemical Formula 2, X¹ and X² may each independently be a halogen or —O-L¹-R³, provided that at least one selected from among X¹ and X² is —O-L¹-R³. Herein, L¹ may be a single bond or a substituted or unsubstituted C1 to C10 alkylene group, and R³ may be a cyano group (—CN), a difluorophosphite group (—OPF₂), a substituted or unsubstituted C1 to C10 alkyl group, a substituted or unsubstituted C2 to C10 alkenyl group, a substituted or unsubstituted C3 to C10 cycloalkyl group, a substituted or unsubstituted C3 to C10 cycloalkenyl group, a substituted or unsubstituted C2 to C10 alkynyl group, a substituted or unsubstituted C3 to C10 cycloalkynyl group, or a substituted or unsubstituted C6 to C20 aryl group. Herein, when X¹ and X² are each —O-L¹-R³ at the same time, R³ may be each independently present, or two R³s may be linked to form a substituted or unsubstituted monocyclic or polycyclic aliphatic ring, a substituted or unsubstituted monocyclic ring (e.g., monocyclic aromatic ring), or a polycyclic aromatic ring.

In one or more embodiments, a rechargeable lithium battery may include a positive electrode including a positive electrode active material; a negative electrode including a negative electrode active material; a separator between the positive electrode and the negative electrode; and the aforementioned electrolyte solution.

The electrolyte solution for a rechargeable lithium battery according to one or more embodiments may form a stable film on the electrode(s) of the rechargeable lithium battery, thereby improving rapid charging characteristics and suppressing an increase in resistance in the battery, securing battery safety and high-temperature reliability under high-voltage driving conditions, and improving capacity characteristics and cycle-life characteristics of the rechargeable lithium battery.

### BRIEF DESCRIPTION OF THE DRAWINGS

The accompanying drawing is included to provide a further understanding of the present disclosure, and is incorporated in and constitutes a part of this specification. The drawing illustrates example embodiments of the present disclosure and, together with the description, serve to explain principles of present disclosure. In the drawing:

The drawing is a schematic view illustrating a rechargeable lithium battery according to one or more embodiments of the present disclosure.

### DETAILED DESCRIPTION

The present disclosure may be modified in many alternate forms, and thus specific embodiments will be exemplified in the drawing and described in more detail. It should be understood, however, that it is not intended to limit the present disclosure to the particular forms disclosed, but rather, is intended to cover all modifications, equivalents, and alternatives falling within the spirit and scope of the present disclosure.

Hereinafter, example embodiments will be described in more detail so that those of ordinary skill in the art may easily implement them. However, the present disclosure may be embodied in many different forms and is not construed as limited to the example embodiments set forth herein.

The terminology utilized herein is utilized to describe embodiments only, and is not intended to limit the present disclosure. As utilized herein, the singular forms “a,” “an,” and “the” are intended to include the plural forms as well, unless the context clearly indicates otherwise. Further, the utilization of “may” when describing embodiments of the present disclosure may refer to “one or more embodiments of the present disclosure”.

As utilized herein, “combination thereof” refers to a mixture, laminate, composite, copolymer, alloy, blend, reaction product, and/or the like of the constituents.

Herein, it should be understood that terms such as “comprise(s),” “include(s),” or “have/has” are intended to designate the presence of an embodied feature, number, step, element, or a combination thereof, but it does not preclude the possibility of the presence or addition of one or more other features, numbers, steps, elements, or a combination thereof.

In the drawings, the thickness of layers, films, panels, regions, etc., may be exaggerated for clarity and like reference numerals designate like elements throughout the present disclosure, and duplicative descriptions thereof may not be provided for conciseness. It will be understood that when an element such as a layer, film, region, or substrate is referred to as being “on” another element, it may be directly on the other element or intervening elements may also be present. In contrast, when an element is referred to as being “directly on” another element, there are no intervening elements present.

In some embodiments, “layer” utilized herein may include not only a shape formed on the whole surface when viewed from a plan view, but also a shape formed on a partial surface.

In some embodiments, an average particle diameter may be measured by a method well suitable to those skilled in the art, for example, may be measured by a particle size analyzer, or may be measured by a transmission electron micrograph or a scanning electron micrograph. In some embodiments, an average particle diameter value may be obtained by measuring utilizing a dynamic light scattering method, performing data analysis, counting the number of particles for each particle size range, and calculating from the data. Unless otherwise defined, an average particle diameter may refer to the diameter (D50) of particles having a cumulative volume of 50 volume % in a particle size distribution. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size. Also, in the present disclosure, when particles are spherical, “diameter” indicates a particle diameter or an average particle diameter, and when the particles are non-spherical, the “diameter” indicates a major axis length or an average major axis length.

Herein, “or” is not to be construed as an exclusive meaning, for example, “A or B” is construed to include A, B, A+B, and/or the like. Further, as used herein, the term “and/or” includes any and all combinations of one or more of the associated listed items. The “/” utilized herein may be interpreted as “and” or as “or” depending on the situation. Expressions such as “at least one of,” “one of,” and “selected from,” when preceding a list of elements, modify the entire list of elements and do not modify the individual elements of the list. For example, “at least one of a, b, or c”, “at least one of a, b, and/or c”, “at least one selected from a, b, and c”, “at least one selected from among a to c”, etc. may indicate only a, only b, only c, both (e.g., simultaneously) a and b, both (e.g., simultaneously) a and c, both (e.g., simultaneously) b and c, all of a, b, and c, or variations thereof.

As utilized herein, unless otherwise defined, “substituted” may refer to replacement of at least one hydrogen in a substituent or a compound by deuterium, a halogen, a hydroxyl group, an amino group, a C1 to C30 amine group, a nitro group, a C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 fluoroalkyl group, a cyano group, or a combination thereof.

For example, in some embodiments, “substituted” may refer to replacement of at least one hydrogen in a substituent or compound by deuterium, a halogen, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C10 fluoroalkyl group, or a cyano group. For example, in some embodiments, “substituted” may refer to replacement of at least one hydrogen in a substituent or compound by deuterium, a halogen group, a C1 to C20 alkyl group, a C6 to C30 aryl group, a C1 to C10 fluoroalkyl group, or a cyano group. In some embodiments, “substituted” may refer to replacement of at least one hydrogen in a substituent or compound by deuterium, a halogen, a C1 to C5 alkyl group, a C6 to C18 aryl group, a C1 to C5 fluoroalkyl group, or a cyano group. For example, in one or more embodiments, “substituted” refers to replacement of at least one hydrogen in a substituent or compound by deuterium, a cyano group, a halogen, a methyl group, an ethyl group, a propyl group, a butyl group, a phenyl group, a biphenyl group, a terphenyl group, a trifluoromethyl group, or a naphthyl group.

### Electrolyte Solution for Rechargeable Lithium Battery

In one or more embodiments, an electrolyte solution for a rechargeable lithium battery may include a non-aqueous organic solvent, a lithium salt, and an additive, wherein the additive may include a first compound (that is CsPF₆, a compound represented by Chemical Formula 1, or a combination thereof), a second compound represented by Chemical Formula 2, and a silver (Ag) salt.

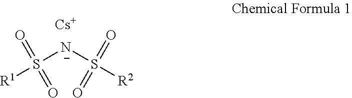

In Chemical Formula 1, R¹ and R² may each independently be a fluoro group or a C1 to C4 fluoroalkyl group substituted with at least one fluoro group.

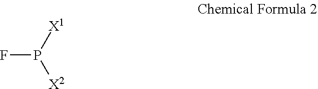

In Chemical Formula 2, X¹ and X² may each independently be a halogen or —O-L¹-R³, and at least one selected from among X¹ and X² may be —O-L¹-R³. Herein, L¹ may be a single bond or a substituted or unsubstituted C1 to C10 alkylene group, R³ may be a cyano group (—CN), a difluorophosphite group (—OPF₂), a substituted or unsubstituted C1 to C10 alkyl group, a substituted or unsubstituted C2 to C10 alkenyl group, a substituted or unsubstituted C3 to C10 cycloalkyl group, a substituted or unsubstituted C3 to C10 cycloalkenyl group, a substituted or unsubstituted C2 to C10 alkynyl group, a substituted or unsubstituted C3 to C10 cycloalkynyl group, or a substituted or unsubstituted C6 to C20 aryl group. Herein, when X¹ and X² are each —O-L¹-R³ at the same time, R³ may be each independently present, or two R³s may be linked to form a substituted or unsubstituted monocyclic or polycyclic aliphatic ring a substituted or unsubstituted monocyclic ring (e.g., monocyclic aromatic ring), or a polycyclic aromatic ring.

When the electrolyte solution for a rechargeable lithium battery is utilized in a rechargeable lithium battery, the electrolyte solution for a rechargeable lithium battery may form a stable film on positive and negative electrodes of the rechargeable lithium battery and thus improve rapid charging characteristics and suppress or reduce a resistance increase in the battery, thereby securing battery safety and high temperature reliability under high voltage driving conditions and resultantly, improving capacity characteristics and cycle-life characteristics of the rechargeable lithium battery.

The electrolyte solution for a rechargeable lithium battery concurrently (e.g., simultaneously) includes the first compound, the second compound, and the Ag salt as additives and thus may effectively solve a problem of forming lithium dendrites on the surface of the negative electrode as well as form a stronger film on the surface of the negative electrode than embodiments of utilizing each compound alone, resultantly improving battery cycle-life characteristics during the rapid charge and concurrently (e.g., simultaneously), effectively suppressing the resistance increase phenomenon caused by the rapid charge.

### Additive

The additive is added to the electrolyte solution to improve battery cycle-life characteristics during the rapid charge and concurrently (e.g., simultaneously), effectively suppress or reduce the resistance increase phenomenon caused by the rapid charge and in addition, to effectively solve the problem of forming lithium dendrites on the surface of the negative electrode.

The additive may include the first compound, the second compound, and the Ag salt, which will be described in more detail later. The additive may be included, for example, in an amount of about 0.1 wt % to about 30.0 wt %, about 1.0 wt % to about 20.0 wt %, or about 2.0 wt % to about 15.0 wt % based on a total weight of the electrolyte solution for a rechargeable lithium battery.

### First Compound

The first compound may be a compound including cesium hexafluorophosphate (CsPF₆), cesium sulfonylimide salt or a combination thereof. The first compound may be decomposed in the electrolyte solution to form a film on the surfaces of positive and negative electrodes to effectively control elution of lithium ions from the positive electrode and thus prevent or reduce decomposition of the positive electrode. For example, the first compound may be reduced and decomposed earlier than a carbonate-based solvent included in the non-aqueous organic solvent and forms an SEI (solid electrolyte interface) film on the negative electrode to prevent or reduce decomposition of the electrolyte solution and the resulting decomposition of the negative electrode, and to suppress an internal resistance increase due to the gas generation. The SEI film on the negative electrode is partially decomposed through a reduction reaction during the charge and discharge, moves toward to the positive electrode surface, and also, forms a film on the positive electrode surface through an oxidation reaction to prevent or reduce decomposition of the positive electrode surface and oxidation reaction of the electrolyte solution, thereby contributing to improvement of high-temperature and low-temperature cycle-life characteristics of the rechargeable lithium battery.

For example, in one or more embodiments, the electrolyte solution for a rechargeable lithium battery may improve cycle-life characteristics and safety of a rechargeable lithium battery by including CsPF₆ and/or a first compound that is a compound represented by Chemical Formula 1.

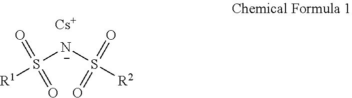

In Chemical Formula 1, R¹ and R² may each independently be a fluoro group or a C1 to C4 fluoroalkyl group substituted with at least one fluoro group.

For example, in some embodiments, R¹ and R² in Chemical Formula 1 may each independently be a fluoro group or a C1 to C4 fluoroalkyl group substituted with at least two fluoro groups.

For example, in some embodiments, R¹ and R² in Chemical Formula 1 may each independently be a fluoro group or a C1 to C4 fluoroalkyl group substituted with at least three fluoro groups.

In one or more embodiments, R¹ and R² in Chemical Formula 1 may each independently be a fluoro group or a C1 to C3 fluoroalkyl group substituted with at least three fluoro groups.

In one or more embodiments, R¹ and R² in Chemical Formula 1 may each independently be a fluoro group or a C1 to C2 fluoroalkyl group substituted with at least three fluoro groups.

In one or more embodiments, the compound represented by Chemical Formula 1 may be represented by Chemical Formula 1-1 or 1-2.

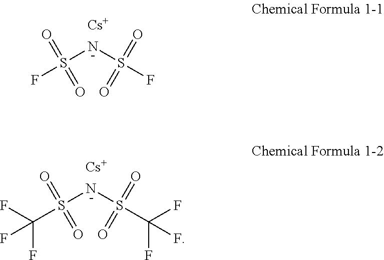

The first compound may be included in an amount of about 0.05 wt % to about 5.0 wt % based on a total weight of the electrolyte solution for a rechargeable lithium battery.

For example, in one or more embodiments, the first compound may be included in an amount of about 0.1 wt % to about 2.5 wt %, about 0.1 wt % to about 2.0 wt %, about 0.1 wt % to about 1.0 wt %, or about 0.2 wt % to about 1.0 wt %, for example about 0.2 wt % to about 0.5 wt %.

When the first compound is included within the above ranges, an increase in internal resistance due to gas generation may be suppressed or reduced, and the rechargeable lithium battery having improved cycle-life characteristics at high temperatures may be implemented.

### Second Compound

The second compound, which is a fluoro phosphite-based compound, may be a material suppressing high-temperature decomposition of the electrolyte solution through stabilization of the lithium salt in the electrolyte solution as well as possessing flame retardant characteristics, thus improving the suppression of the gas generation in the rechargeable lithium battery at a high temperature and also improving battery safety and cycle-life characteristics at the same time.

The second compound may be represented by Chemical Formula 2.

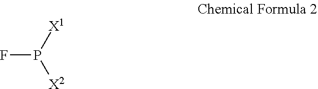

In Chemical Formula 2, X¹ and X² may each independently be a halogen or —O-L¹-R³,

at least one selected from among X¹ and X² may be —O-L¹-R³,

L¹ may be a single bond or a substituted or unsubstituted C1 to C10 alkylene group,

R³ may be a cyano group (—CN), a difluorophosphite group (—OPF₂), a substituted or unsubstituted C1 to C10 alkyl group, a substituted or unsubstituted C2 to C10 alkenyl group, or a substituted or unsubstituted C3 to C10 cycloalkyl group, a substituted or unsubstituted C3 to C10 cycloalkenyl group, a substituted or unsubstituted C2 to C10 alkynyl group, a substituted or unsubstituted C3 to C10 cycloalkynyl group, or a substituted or unsubstituted C6 to C20 aryl group, and

when X¹ and X² are each —O-L¹-R³ at the same time, R³ may be each independently present, or two R³s may be linked to form a substituted or unsubstituted monocyclic or polycyclic aliphatic ring, or a substituted or unsubstituted monocyclic ring (e.g., monocyclic aromatic ring), or a polycyclic aromatic ring.

For example, in one or more embodiments, one of X¹ and X² in Chemical Formula 2 may be a fluoro group, and the other may be —O-L²-R⁴, wherein L² may be a single bond or a substituted or unsubstituted C1 to C10 alkylene group and R⁴ may be a cyano group (—CN) or a difluorophosphite group (—OPF₂).

For example, in one or more embodiments, the second compound may be represented by Chemical Formula 2, and Chemical Formula 2 may be represented by Chemical Formula 2-1.

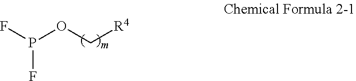

In Chemical Formula 2-1,

m may be an integer of 1 to 5, and

R⁴ may be a cyano group (—CN) or a difluorophosphite group (—OPF₂).

In one or more embodiments, in Chemical Formula 2, X¹ may be —O-L³-R⁵, X² may be —O-L⁴-R⁶, L³ and L⁴ may each independently be a single bond or a substituted or unsubstituted C1 to C10 alkylene group, R⁵ and R⁶ may each independently be a substituted or unsubstituted C1 to C10 alkyl group, and/or R⁵ and R⁶ may be linked to form a substituted or unsubstituted monocyclic or polycyclic aliphatic ring.

For example, in some embodiments, the second compound may be represented by Formula 2-2.

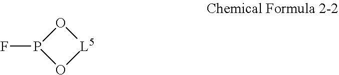

In Chemical Formula 2-2, L⁵ is a substituted or unsubstituted C2 to C5 alkylene group.

In some embodiments, the second compound may be represented by Chemical Formula 2-2a or 2-2b.

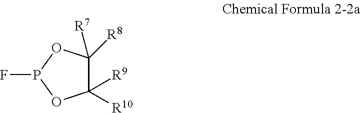

In Chemical Formula 2-2a, R⁷ to R¹⁰ may each independently be hydrogen, a halogen, or a substituted or unsubstituted C1 to C5 alkyl group.

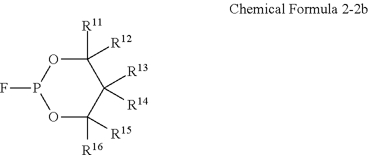

In Chemical Formula 2-2b, R¹¹ to R¹⁶ may each independently be hydrogen, a halogen, or a substituted or unsubstituted C1 to C5 alkyl group.

For example, in some embodiments, the second compound may be any one selected from the compounds listed in Group 1.

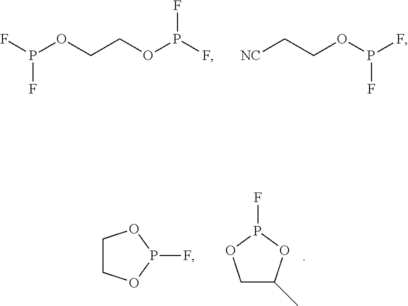

The second compound may be included in an amount of about 0.1 wt % to about 10 wt % based on a total weight of the electrolyte solution for a rechargeable lithium battery. For example, in one or more embodiments, the second compound may be included in an amount of about 0.2 wt % to about 10 wt %, about 0.5 wt % to about 10 wt %, about 1.0 wt % to about 10 wt %, or about 1.0 wt % to about 5.0 wt %. When the second compound is included in the above ranges, the rechargeable lithium battery with improved battery safety and lifespan characteristics may be implemented.

### Ag Salt

The Ag salt improves the conductivity of lithium ions in the negative electrode of the rechargeable lithium battery and suppresses the growth of lithium dendrites generated on the surface of the negative electrode. Accordingly, the Ag salt is a material reducing a side reaction between the electrodes and the electrolyte solution and improving safety of the rechargeable lithium battery (increasing cycle-life of the battery and suppressing gas generation).

The Ag salt may include one or more selected from AgNO₃, AgNO₂, AgN₃, AgCN, AgPF₆, AgN(FSO₂)₂ (AgFSI), AgN(CF₃SO₂)₂ (AgTFSI), AgF, AgSO₃CF₃, and AgBF₄, but embodiments of the present disclosure are not limited thereto.

For example, in some embodiments, the Ag salt may include AgNO₃, AgNO₂, AgN₃ and/or AgCN. In one embodiment, the Ag salt may be AgNO₃.

The Ag salt may be included in an amount of about 0.1 wt % to about 10 wt % based on a total weight of the electrolyte solution for a rechargeable lithium battery. For example, in one or more embodiments, the Ag salt may be included in an amount of about 0.2 wt % to about 10 wt %, about 0.5 wt % to about 10 wt %, or about 1.0 wt % to about 10 wt %, or for example about 1.0 wt % to about 5.0 wt %. When the Ag salt is included in the above ranges, a growth of lithium dendrites is effectively inhibited, so that the rechargeable lithium battery with improved battery safety may be realized.

According to one or more embodiments, the additive included in the electrolyte solution for a rechargeable lithium battery may be a composition including cesium bis(fluorosulfonyl)imide as the first compound, at least one selected from the compounds listed in Group 1, and AgNO₃ as the Ag salt.

According to one or more embodiments, the additive may be a composition including cesium bis(trifluoromethanesulfonyl)imide as the first compound, at least one selected from the compounds listed in Group 1 as the second compound, and AgNO₃ as the Ag salt.

For example, in one or more embodiments, the first compound and the second compound may be included in a weight ratio of about 5:95 to about 40:60. In some embodiments, the first compound and the second compound may be included in a weight ratio of about 5:95 to about 30:70, for example, about 5:95 to about 20:80.

For example, in one or more embodiments, the first compound and the Ag salt may be included in a weight ratio of about 30:70 to about 70:30. In some embodiments, the first compound and the Ag salt may be included in a weight ratio of about 30:70 to about 60:40, for example, about 30:70 to about 50:50.

For example, in one or more embodiments, the Ag salt and the second compound may be included in a weight ratio of about 5:95 to about 40:60. In some embodiments, the Ag salt and the second compound may be included in a weight ratio of about 10:90 to about 40:60, for example, about 10:90 to about 30:70.

When the first compound, the second compound, and the Ag salt are included within the aforementioned weight ratios, rapid charging characteristics may be improved, a resistance increase in the battery is suppressed or reduced, thus securing battery safety and high-temperature reliability under high voltage-driving conditions and resultantly, realizing a rechargeable lithium battery with improved capacity characteristics and cycle-life characteristics.

### Third Compound

In one or more embodiments, the additive may further include a third compound.

The third compound has a structure including a cesium fluorosulfonylimide salt, and is decomposed in the electrolyte solution to form films on the surfaces of the positive and negative electrodes, respectively. For example, the film on the positive electrode surface may effectively control elution of lithium ions from the positive electrode and thus prevent or reduce decomposition of the positive electrode.

In some embodiments, the third compound is earlier reduced and decomposed than a carbonate-based solvent included in the non-aqueous organic solvent and thus may form an SEI (solid electrolyte interface) film on the negative electrode to prevent or reduce decomposition of the electrolyte solution and the resulting decomposition of the negative electrode, thereby suppressing an increase in internal resistance due to the gas generation. The SEI film formed on the negative electrode is partially decomposed through a reduction reaction during the charge and discharge, moves toward the positive electrode surface, and also forms a film on the positive electrode surface through an oxidation reaction to prevent or reduce decomposition of the positive electrode surface and the resulting oxidation reaction of the electrolyte solution, resultantly contributing to improvement of high-temperature and low-temperature cycle-life characteristics of the rechargeable lithium battery.

The third compound may be represented by Chemical Formula 3.

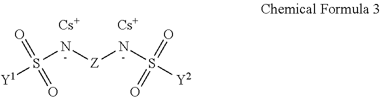

In Chemical Formula 3, Z is C(═O) or S(═O)₂, and Y¹ and Y² may each independently be a fluoro group or a C1 to C5 fluoroalkyl group substituted with at least one fluoro group.

For example, in one or more embodiments, the third compound may be represented by any one selected from Chemical Formulas 3-1 to 3-8.

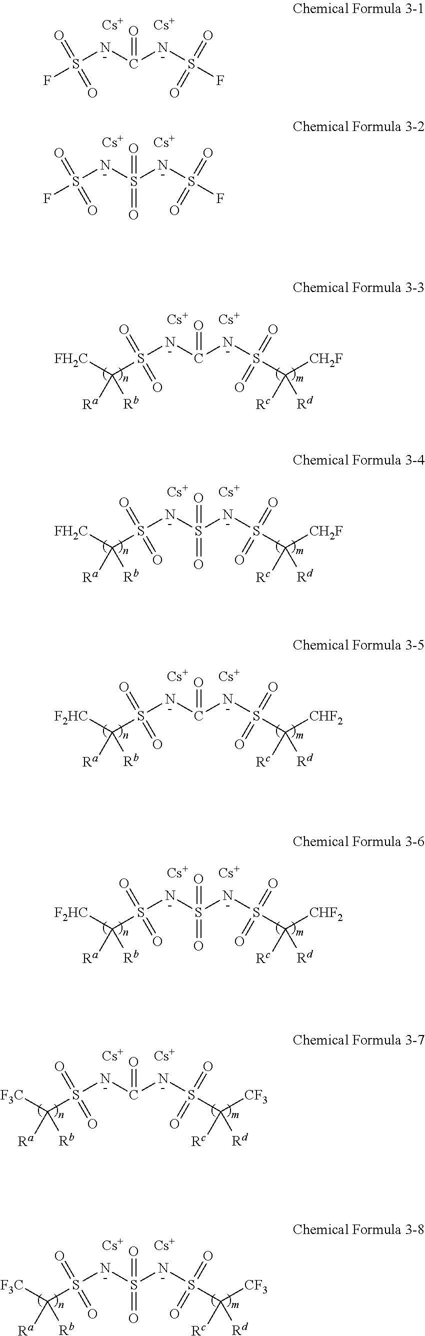

In Chemical Formulas 3-3 to 3-8, R^{a}, R^{b}, R^{c}, and R^{d} may each independently be hydrogen or a fluoro group, and n and m may each independently be an integer of 0 to 4.

For example, in some embodiments, the third compound may be represented by Chemical Formula 3-1 or 3-2.

The third compound may be included in an amount of about 0.05 wt % to about 5 wt % based on a total weight of the electrolyte solution for a rechargeable lithium battery. For example, in one or more embodiments, the third compound may be included in an amount of about 0.1 wt % to about 5.0 wt %, about 0.2 wt % to about 5.0 wt %, or about 0.5 wt % to about 5.0 wt %, or for example about 0.5 wt % to about 2.5 wt %. When the third compound is included within the above ranges, the rechargeable lithium battery having improved lifespan characteristics and low-temperature output characteristics may be implemented by preventing or reducing long-term charge/discharge or resistance increase at low temperatures.

### Other Additives

In one or more embodiments, the electrolyte solution for a rechargeable lithium battery may further include other additives other than those described above. When the other additives are further included, high-temperature storage characteristics may be improved, such as effectively controlling gas generated from the positive electrode and the negative electrode during high-temperature storage.

The other additives may include one or more selected from vinylene carbonate (VC), fluoroethylene carbonate (FEC), difluoroethylene carbonate (DFEC), chloroethylene carbonate (CEC), dichloroethylene carbonate (DCEC), bromoethylene carbonate (BEC), dibromoethylene carbonate (DBEC), nitroethylene carbonate, cyanoethylene carbonate, vinylethylene carbonate (VEC), succinonitrile (SN), adiponitrile (AN), 1,3,6-hexanetricyanide (HTCN)), propene sultone (PST), propane sultone (PS), lithium tetrafluoroborate (LiBF₄), lithium difluorophosphate (LiPO₂F₂), and 2-fluoro biphenyl (2-FBP), but embodiments of the present disclosure are not limited thereto.

The other additives may be included in an amount of about 0.2 wt % to about 20 wt % based on a total weight of the electrolyte solution for a rechargeable lithium battery. For example, in one or more embodiments, the other additives may be included in an amount of about 0.2 wt % to about 15 wt %, for example about 0.2 wt % to about 10 wt %. When the other additives are included within the ranges, a rechargeable lithium battery with improved storage characteristics at a high temperature such as effectively controlling gas generated from the positive and negative electrodes and/or the like may be realized.

### Non-aqueous Organic Solvent

The non-aqueous organic solvent serves as a medium for transmitting ions taking part in the electrochemical reaction of a battery.

The non-aqueous organic solvent may include a carbonate-based, ester-based, ether-based, ketone-based, alcohol-based, or aprotic solvent.

The carbonate-based solvent may include dimethyl carbonate (DMC), diethyl carbonate (DEC), dipropyl carbonate (DPC), methylpropyl carbonate (MPC), ethylpropyl carbonate (EPC), methylethyl carbonate (MEC), ethylene carbonate (EC), propylene carbonate (PC), butylene carbonate (BC), and/or the like.

The ester-based solvent may include methyl acetate, ethyl acetate, n-propyl acetate, dimethylacetate, methylpropionate, ethylpropionate, γ-butyrolactone, decanolide, valerolactone, mevalonolactone, caprolactone, and/or the like.

The ether-based solvent may include dibutyl ether, tetraglyme, diglyme, dimethoxyethane, 2-methyltetrahydrofuran, tetrahydrofuran, and/or the like, and

the ketone-based solvent may include cyclohexanone, and/or the like.

In some embodiments, the alcohol-based solvent may include ethanol, isopropyl alcohol, and/or the like, and

the aprotic solvent may include nitriles such as R—CN (wherein, R is a C2 to C20 linear, branched, or cyclic hydrocarbon group, and may include a double bond, an aromatic ring, or an ether bond), and/or the like, amides such as dimethyl formamide, and/or the like, dioxolanes such as 1,3-dioxolane, sulfolanes, and/or the like.

The non-aqueous organic solvent may be utilized alone or in a mixture. When the non-aqueous organic solvent is utilized in a mixture, a mixture ratio may be controlled or selected in accordance with a desirable battery performance.

In some embodiments, the carbonate-based solvent may include a mixture of a cyclic carbonate and a chain carbonate. In these embodiments, when the cyclic carbonate and the chain carbonate are mixed in a volume ratio of about 1:1 to about 1:9, the electrolyte solution may exhibit excellent or suitable performance.

In some embodiments, the non-aqueous organic solvent may further include an aromatic hydrocarbon-based organic solvent in the carbonate-based solvent. In these embodiments, the carbonate-based solvent and the aromatic hydrocarbon-based organic solvent may be mixed in a volume ratio of about 1:1 to about 30:1.

As the aromatic hydrocarbon-based solvent, an aromatic hydrocarbon-based compound represented by Chemical Formula I may be utilized.

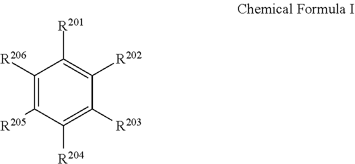

In Chemical Formula I, R²⁰¹ to R²⁰⁶ may be the same or different and may each independently be selected from hydrogen, a halogen, a C1 to C10 alkyl group, a C1 to C10 haloalkyl group, and a combination thereof.

Non-limiting examples of the aromatic hydrocarbon-based solvent may be selected from benzene, fluorobenzene, 1,2-difluorobenzene, 1,3-difluorobenzene, 1,4-difluorobenzene, 1,2,3-trifluorobenzene, 1,2,4-trifluorobenzene, chlorobenzene, 1,2-dichlorobenzene, 1,3-dichlorobenzene, 1,4-dichlorobenzene, 1,2,3-trichlorobenzene, 1,2,4-trichlorobenzene, iodobenzene, 1,2-diiodobenzene, 1,3-diiodobenzene, 1,4-diiodobenzene, 1,2,3-triiodobenzene, 1,2,4-triiodobenzene, toluene, fluorotoluene, 2,3-difluorotoluene, 2,4-difluorotoluene, 2,5-difluorotoluene, 2,3,4-trifluorotoluene, 2,3,5-trifluorotoluene, chlorotoluene, 2,3-dichlorotoluene, 2,4-dichlorotoluene, 2,5-dichlorotoluene, 2,3,4-trichlorotoluene, 2,3,5-trichlorotoluene, iodotoluene, 2,3-diiodotoluene, 2,4-diiodotoluene, 2,5-diiodotoluene, 2,3,4-triiodotoluene, 2,3,5-triiodotoluene, xylene, and a combination thereof.

In one or more embodiments, the electrolyte solution may further include vinylene carbonate or an ethylene carbonate-based compound of Chemical Formula II in order to improve cycle-life of a battery.

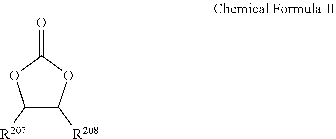

In Chemical Formula II, R²⁰⁷ and R²⁰⁸ may be the same or different, and may each independently be selected from hydrogen, a halogen, a cyano group, a nitro group, and fluorinated C1 to C5 alkyl group, provided that at least one selected from R²⁰⁷ and R²⁰⁸ is selected from a halogen, a cyano group, a nitro group, and fluorinated C1 to C5 alkyl group, but both of R²⁰⁷ and R²⁰⁸ are not simultaneously hydrogen.

Non-limiting examples of the ethylene-based carbonate-based compound may be difluoro ethylenecarbonate, chloroethylene carbonate, dichloroethylene carbonate, bromoethylene carbonate, dibromoethylene carbonate, nitroethylene carbonate, cyanoethylene carbonate, and/or fluoroethylene carbonate. The amount of the additive for improving cycle-life may be utilized within an appropriate or suitable range.

### Lithium Salt

The lithium salt dissolved in the non-aqueous organic solvent supplies lithium ions in a battery (e.g., rechargeable lithium battery), enables a basic operation of a rechargeable lithium battery, and improves transportation of lithium ions between positive and negative electrodes of the rechargeable lithium battery.

Non-limiting examples of the lithium salt may include at least one selected from LiPF₆, LiBF₄, LiSbF₆, LiAsF₆, LIN(SO₂C₂F₅)₂, Li(CF₃SO₂)₂N, LIN(SO₃C₂F₅)₂, Li(FSO₂)₂N (lithium bis(fluorosulfonyl)imide, LiFSI), LiC₄F₉SO₃, LiCIO₄, LiAIO₂, LiAICI₄, LIPO₂F₂, LIN(CₓF₂ₓ₊₁SO₂)(C_{y}F_{2y+1}SO₂) (wherein x and y are natural numbers, for example, an integer of 1 to 20), lithium difluoro(bisoxalato) phosphate, LiCI, Lil, LiB(C₂O₄)₂ (lithium bis(oxalato) borate, LiBOB), and lithium difluoro(oxalato)borate (LiDFOB).

The lithium salt may be utilized in a concentration in a range of about 0.1 M to about 2.0 M. When the lithium salt is included at the above concentration range, the electrolyte solution may have excellent or suitable performance and lithium ion mobility due to optimal or suitable conductivity and viscosity of the electrolyte solution.

### Rechargeable Lithium Battery

In one or more embodiments, a rechargeable lithium battery may include a positive electrode including a positive electrode active material, a negative electrode including a negative electrode active material, a separator between the positive electrode and the positive electrode, and the aforementioned electrolyte solution.

The drawing is a schematic view illustrating a rechargeable lithium battery according to one or more embodiments of the present disclosure. Referring to the drawing, the rechargeable lithium battery 100 may include a battery cell including a positive electrode 114, a negative electrode 112 facing the positive electrode 114, a separator 113 between the positive electrode 114 and the negative electrode 112, and an electrolyte solution for a rechargeable lithium battery impregnating the positive electrode 114, the negative electrode 112, and the separator 113, a battery case 120 containing the battery cell, and a sealing member 140 sealing the battery case 120.

### Positive Electrode

The positive electrode may include a positive electrode current collector and a positive electrode active material layer positioned on the positive electrode current collector. The positive electrode active material layer may include a positive electrode active material, and may optionally further include a binder and/or a conductive material.

The positive electrode active material may be applied without limitation as long as it is generally utilized in a rechargeable lithium battery. For example, in one or more embodiments, the positive electrode active material may be a compound capable of intercalating and deintercalating lithium, and may include a compound represented by any one selected from the following chemical formulas:
LiₐA_{1-b}X_{b}D₂ (0.90≤a≤1.8, 0≤b≤0.5);
LiₐA_{1-b}X_{b}O_{2-c}D_{c} (0.90≤a≤1.8, 0≤b≤0.5, 0≤c≤0.05);
LiₐE_{1-b}X_{b}O_{2-c}D_{c} (0.90≤a≤1.8, 0≤b≤0.5, 0≤c≤0.05);
LiₐE_{2-b}X_{b}O_{4-c}D_{c} (0.90≤a≤1.8, 0≤b≤0.5, 0≤c≤0.05);
LiₐNi_{1-b-c}CO_{b}X_{c}Dₐ (0.90≤a≤1.8, 0≤b≤0.5, 0≤c≤0.5, 0≤a≤2);
LiₐNi_{1-b-c}CO_{b}X_{c}O₂₋ₐTₐ (0.90≤a≤1.8, 0≤b≤0.5, 0≤c≤0.05, 0≤a≤2);
LiₐNi_{1-b-c}CO_{b}X_{c}O₂₋ₐT₂ (0.90≤a≤1.8, 0≤b≤0.5, 0≤c≤0.05, 0≤a≤2);
LiₐNi_{1-b-c}Mn_{b}X_{c}Dₐ (0.90≤a≤1.8, 0≤b≤0.5, 0<<≤0.05, 0≤a≤2);
LiₐNi_{1-b-c}Mn_{b}X_{c}O₂₋ₐTₐ (0.90≤a≤1.8, 0≤b≤0.5, 0≤c≤0.05, 0≤a≤2);
LiₐNi_{1-b-c}Mn_{b}X_{c}O₂₋ₐT₂ (0.90≤a≤1.8, 0≤b≤0.5, 0≤c≤0.05, 0≤a≤2);
LiₐNi_{D}E_{c}G_{d}O₂ (0.90≤a≤1.8, 0≤b≤0.9, 0≤c≤0.5, 0.001≤d≤0.1);
LiₐNi_{b}Co_{c}Mn_{d}GeO₂(0.90≤a≤1.8, 0≤b≤0.9, 0≤c≤0.5, 0≤d≤0.5, 0.001≤e≤0.1);
LiₐNiG_{b}O₂ (0.90≤a≤1.8, 0.001≤b≤0.1);
LiₐCoG_{b}O₂(0.90≤a≤1.8, 0.001≤b≤0.1);
LiₐMn_{1-b}G_{b}O₂ (0.90≤a≤1.8, 0.001≤b≤0.1);
LiₐMn₂G_{b}O₄ (0.90≤a≤1.8, 0.001≤b≤0.1);
LiₐMn_{1-g}G_{g}PO₄ (0.90≤a≤1.8, 0≤g≤0.5);
QO₂; QS₂; LiQS₂;
V₂O₅; LIV₂O₅;
LiZO₂;
LiNIVO₄;
Li₍₃₋₁₎J₂(PO₄)₃ (0≤f≤2);
Li₍₃₋₁₎Fez(PO₄)₃ (0≤f≤2); and
LiaFePO₄ (0.90≤a≤1.8).

In chemical formulas, A may be selected from nickel (Ni), cobalt (Co), manganese (Mn), and a combination thereof; X may be selected from aluminum (Al), Ni, Co, Mn, chromium (Cr), iron (Fe), magnesium (Mg), strontium (Sr), vanadium (V), a rare earth element, and a combination thereof; D is selected from oxygen (O), fluorine (F), sulfur (S), phosphorus (P), and a combination thereof; E is selected from Co, Mn, and a combination thereof; T is selected from F, S, P, and a combination thereof; G is selected from Al, Cr, Mn, Fe, Mg, lanthanum (La), cerium (Ce), Sr, V, and a combination thereof; Q is selected from titanium (Ti), Mo, Mn, and a combination thereof; Z is selected from Cr, V, Fe, scandium (Sc), yttrium (Y), and a combination thereof; and J is selected from V, Cr, Mn, Co, Ni, copper (Cu), and a combination thereof.

The positive electrode active material may be, for example, lithium cobalt oxide (LCO), lithium nickel oxide (LNO), lithium nickel cobalt oxide (NC), lithium nickel cobalt aluminum oxide (NCA), lithium nickel cobalt manganese oxide (NCM), lithium nickel manganese oxide (NM), lithium manganese oxide (LMO), lithium iron phosphate (LFP), and/or the like.

In one or more embodiments, the positive electrode active material may include a lithium cobalt-based oxide. A positive electrode utilizing lithium cobalt-based oxide as a positive electrode active material may suppress or reduce battery resistance and improve overall battery performance by exhibiting a synergistic effect in a 4.5 V high voltage design or rapid charging system when utilized with the aforementioned electrolyte solution.

The lithium cobalt-based oxide, for example, may be represented by Chemical Formula 4:

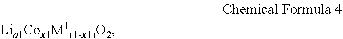

wherein, in Chemical Formula 4, 0.9≤a1≤1.8, and 0.7≤x1≤1, and M1 may be at least one element selected from aluminum (Al), boron (B), barium (Ba), calcium (Ca), cerium (Ce), chromium (Cr), copper (Cu), fluorine (F), iron (Fe), magnesium (Mg), manganese (Mn), molybdenum (Mo), nickel (Ni), phosphorus (P), sulfur (S), selenium (Se), silicon (Si), strontium (Sr), titanium (Ti), vanadium (V), tungsten (W), yttrium (Y), zinc (Zn), and zirconium (Zr).

In Chemical Formula 4, x1 represents a molar content (e.g., amount) of cobalt and may be, for example, 0.8≤x1≤1, 0.9≤x1≤1, or 0.95≤x1≤1.

The positive electrode active material may have an average particle diameter (D50) of about 1 μm to about 25 μm, for example about 3 μm to about 25 μm, about 5 μm to about 25 μm, about 5 μm to about 20 μm, about 8 μm to about 20 μm, or about 10 μm to about 18 μm. The positive electrode active material having such a particle size range may be harmoniously mixed with other components in the positive electrode active material layer and may realize high capacity and high energy density. Herein, the average particle diameter (D50) is measured by a particle size analyzer utilizing a laser diffraction method, and refers to a diameter of particles having a cumulative volume of 50 volume % in a particle size distribution.

In one or more embodiments, the positive electrode active material may be in the form of secondary particles in which a plurality of primary particles are aggregated, or may be in the form of single particles. In some embodiments, the positive electrode active material may have a spherical or near-spherical shape, or may have a polyhedral or irregular shape.

In one or more embodiments, the positive electrode active material layer may include a binder. The binder improves binding properties of positive electrode active material particles with one another and with the positive electrode current collector, and non-limiting examples thereof may be polyvinyl alcohol, carboxylmethyl cellulose, hydroxypropyl cellulose, diacetyl cellulose, polyvinylchloride, carboxylated polyvinylchloride, polyvinylfluoride, an ethylene oxide-containing polymer, polyvinylpyrrolidone, polyurethane, polytetrafluoroethylene, polyvinylidene fluoride, polyethylene, polypropylene, a styrene-butadiene rubber, an acrylated styrene-butadiene rubber, an epoxy resin, nylon, and/or the like, but embodiments of the present disclosure are not limited thereto.

A content (e.g., amount) of the binder in the positive electrode active material layer may be about 0.5 wt % to about 5 wt % based on a total weight of the positive electrode active material layer.

In one or more embodiments, the positive electrode active material layer may include a conductive material. The conductive material may be included to provide electrode conductivity, and any electrically conductive material may be utilized as a conductive material unless it causes a chemical change. The conductive material may include a carbon-based material such as natural graphite, artificial graphite, carbon black, acetylene black, ketjen black, a carbon fiber, a carbon nanofiber, a carbon nanotube, and/or the like; a metal-based material of a metal powder or a metal fiber including copper, nickel, aluminum, silver, and/or the like; a conductive polymer such as a polyphenylene derivative; or a mixture thereof.

A content (e.g., amount) of the conductive material in the positive electrode active material layer may be about 1 wt % to about 5 wt % based on a total weight of the positive electrode active material layer.

In one or more embodiments, an aluminum foil may be utilized as the positive electrode current collector, but embodiments of the present disclosure are not limited thereto.

### Negative Electrode

In one or more embodiments, the negative electrode for the rechargeable lithium battery may include a negative electrode current collector and a negative electrode active material layer on the negative electrode current collector. The negative electrode active material layer may include a negative electrode active material, and may further include a binder and/or a conductive material.

The negative electrode active material may include a material that reversibly intercalates/deintercalates lithium ions, a lithium metal, a lithium metal alloy, a material capable of doping/dedoping lithium, and/or a transition metal oxide.

The negative electrode active material may include a material that reversibly intercalates/deintercalates lithium ions, for example, may be crystalline carbon, amorphous carbon, or a combination thereof. The crystalline carbon may be irregular-shaped, sheet, flake, spherical, or fiber-shaped natural graphite or artificial graphite. The amorphous carbon may be a soft carbon, a hard carbon, a mesophase pitch carbonization product, calcined coke, and/or the like.

The lithium metal alloy includes an alloy of lithium and a metal selected from sodium (Na), potassium (K), rubidium (Rb), cesium (Cs), francium (Fr), beryllium (Be), magnesium (Mg), calcium (Ca), strontium (Sr), silicon (Si), antimony (Sb), lead (Pb), indium (In), zinc (Zn), barium (Ba), radium (Ra), germanium (Ge), aluminum (Al), and tin (Sn).

The material capable of doping/dedoping lithium may be a Si-based negative electrode active material or a Sn-based negative electrode active material. The Si-based negative electrode active material may include silicon, a silicon-carbon composite, SiOₓ (0≤x≤2), a Si-Q alloy (wherein Q is an alkali metal, an alkaline-earth metal, a Group 13 element, a Group 14 element, a Group 15 element, a Group 16 element, a transition metal, a rare earth element, or a combination thereof, but not Si), and the Sn-based negative electrode active material may include Sn, SnO₂, Sn—R alloy (wherein R is an alkali metal, an alkaline-earth metal, a Group 13 element, a Group 14 element, a Group 15 element, a Group 16 element, a transition metal, a rare earth element, or a combination thereof, but not Sn). At least one of these materials may be mixed with SiO₂. The elements Q and R may be selected from Mg, Ca, Sr, Ba, Ra, Sc, Y, Ti, Zr, hafnium (Hf), rutherfordium (Rf), V, niobium (Nb), tantalum (Ta), dubnium (db), Cr, Mo, W, seaborgium (Sg), technetium (Tc), rhenium (Re), bohrium (Bh), Fe, Pb, ruthenium (Ru), osmium (Os), hassium (Hs), rhodium (Rh), iridium (Ir), palladium (Pd), platinum (Pt), Cu, silver (Ag), gold (Au), Zn, cadmium (Cd), B, Al, gallium (Ga), Sn, In, thallium (Ti), Ge, P, arsenic (As), Sb, bismuth (Bi), S, Se, tellurium (Te), polonium (Po), and a combination thereof.

In one or more embodiments, the silicon-carbon composite may be, for example, a silicon-carbon composite including a core including crystalline carbon and silicon particles and an amorphous carbon coating layer disposed on the surface of the core. The crystalline carbon may be artificial graphite, natural graphite, or a combination thereof. The amorphous carbon precursor may be a coal-based pitch, mesophase pitch, petroleum-based pitch, coal-based oil, petroleum-based heavy oil, or a polymer resin such as a phenol resin, a furan resin, or a polyimide resin. In these embodiments, a content (e.g., amount) of silicon may be about 10 wt % to about 50 wt % based on a total weight of the silicon-carbon composite. In some embodiments, a content (e.g., amount) of the crystalline carbon may be about 10 wt % to about 70 wt % based on a total weight of the silicon-carbon composite, and a content (e.g., amount) of the amorphous carbon may be about 20 wt % to about 40 wt % based on a total weight of the silicon-carbon composite. In some embodiments, a thickness of the amorphous carbon coating layer may be about 5 nm to about 100 nm. An average particle diameter (D50) of the silicon particles may be about 10 nm to about 20 μm. In some embodiments, the average particle diameter (D50) of the silicon particles may be about 10 nm to about 200 nm. In some embodiments, the silicon particles may exist in an oxidized form, and in these embodiments, an atomic content (e.g., amount) ratio of Si:O in the silicon particles indicating a degree of oxidation may be a weight ratio of about 99:1 to about 33:67. In some embodiments, the silicon particles may be SiOₓ particles, and in these embodiments, the range of x in SiOₓ may be greater than about 0 and less than about 2. In the present disclosure, unless otherwise defined, an average particle diameter (D50) indicates a particle where an accumulated volume is about 50 volume % in a particle size distribution.

In one or more embodiments, the Si-based negative electrode active material or Sn-based negative electrode active material may be mixed with the carbon-based negative electrode active material. When the Si-based negative electrode active material or Sn-based negative electrode active material and the carbon-based negative electrode active material are mixed and utilized, a mixing ratio may be a weight ratio of about 1:99 to about 90:10.

In the negative electrode active material layer, the negative electrode active material may be included in an amount of about 95 wt % to about 99 wt % based on a total weight of the negative electrode active material layer.

In one or more embodiments, the negative electrode active material layer may further include a binder, and may optionally further include a conductive material. A content (e.g., amount) of the binder in the negative electrode active material layer may be about 1 wt % to about 5 wt % based on a total weight of the negative electrode active material layer. In some embodiments, when the conductive material is further included, the negative electrode active material layer may include about 90 wt % to about 98 wt % of the negative electrode active material, about 1 wt % to about 5 wt % of the binder, and about 1 wt % to about 5 wt % of the conductive material.

The binder serves to well adhere the negative electrode active material particles to each other and also to adhere the negative electrode active material to the negative electrode current collector. The binder may be a water-insoluble binder, a water-soluble binder, or a combination thereof.

Non-limiting examples of the water-insoluble binder may include polyvinyl chloride, carboxylated polyvinyl chloride, polyvinyl fluoride, an ethylene oxide-containing polymer, an ethylene propylene copolymer, polystyrene, polyvinylpyrrolidone, polyurethane, polytetrafluoro ethylene, polyvinylidene fluoride, polyethylene, polypropylene, polyamideimide, polyimide, or a combination thereof.

The water-soluble binder may include a rubber binder or a polymer resin binder. The rubber binder may be selected from a styrene-butadiene rubber, an acrylated styrene-butadiene rubber, an acrylonitrile-butadiene rubber, an acrylic rubber, a butyl rubber, a fluororubber, and a combination thereof. The polymer resin binder may be selected from polyethylene oxide, polyvinylpyrrolidone, polyepichlorohydrin, polyphosphazene, polyacrylonitrile, an ethylene propylene diene copolymer, polyvinylpyridine, chlorosulfonated polyethylene, latex, a polyester resin, an acrylic resin, a phenol resin, an epoxy resin, polyvinyl alcohol, and a combination thereof.

When a water-soluble binder is utilized as the binder for the negative electrode, a cellulose-based compound capable of imparting viscosity may be further included as a kind of thickener. As the cellulose-based compound, one or more of carboxymethyl cellulose, hydroxypropylmethyl cellulose, methyl cellulose, or alkali metal salts thereof may be mixed and utilized. The alkali metal may be Na, K, or Li. A content (e.g., amount) of the thickener utilized may be about 0.1 parts by weight to about 3 parts by weight based on 100 parts by weight of the negative electrode active material.

The conductive material may be included to provide electrode conductivity, and any electrically conductive material may be utilized as a conductive material unless it causes a chemical change. Non-limiting examples of the conductive material may include a carbon-based material such as natural graphite, artificial graphite, carbon black, acetylene black, ketjen black, a carbon fiber, a carbon nanofiber, a carbon nanotube, and/or the like; a metal-based material of a metal powder or a metal fiber including copper, nickel, aluminum, silver, and/or the like; a conductive polymer such as a polyphenylene derivative; or a mixture thereof.

The negative electrode current collector may include one selected from a copper foil, a nickel foil, a stainless steel foil, a titanium foil, a nickel foam, a copper foam, a polymer substrate coated with a conductive metal, and a combination thereof.

### Separator

The separator 113 separates the positive electrode 114 and the negative electrode 112 and provides a transporting passage for lithium ions and may be any generally-utilized separator in a lithium ion battery. The separator may have low resistance to ion transport and excellent or suitable impregnation for the electrolyte solution. For example, in one or more embodiments, the separator may include a glass fiber, polyester, polyethylene, polypropylene, polytetrafluoroethylene, or a combination thereof and may have a form of a non-woven fabric or a woven fabric. For example, in some embodiments, in a lithium ion battery (e.g., rechargeable lithium battery), a polyolefin-based polymer separator such as polyethylene and polypropylene is mainly utilized. In order to ensure the heat resistance or mechanical strength, a coated separator including a ceramic component or a polymer material may be utilized. In some embodiments, it may have a mono-layered or multi-layered structure.

Rechargeable lithium batteries may be classified as lithium ion batteries, lithium ion polymer batteries, or lithium polymer batteries according to the presence of a separator and the type or kind of electrolyte solution utilized therein. The rechargeable lithium batteries may have a variety of shapes and sizes, and non-limiting shape examples thereof may include cylindrical, prismatic, coin, or pouch-type or kind batteries, and non-limiting size examples thereof may be thin film batteries or may be rather bulky in size. Structures and manufacturing methods for these batteries pertaining to the present disclosure are well suitable in the art.

The rechargeable lithium battery according to one or more embodiments may be utilized in an electric vehicle (EV), a hybrid electric vehicle such as a plug-in hybrid electric vehicle (PHEV), and/or portable electronic device because it implements a high capacity and has excellent or suitable storage stability, cycle-life characteristics, and high rate characteristics at high temperatures.

Hereinafter, examples of the present disclosure and comparative examples are described in more detail. However, the examples are for the purpose of illustration and are not to be construed as limiting the present disclosure.

### Example 1

### 1. Preparation of Electrolyte Solution

(Hereinafter, in a composition of the electrolyte solution, “wt %” is based on a total content (e.g., amount) of the electrolyte solution (a lithium salt+a non-aqueous organic solvent+additives+other additives, etc.))

A basic electrolyte solution was prepared by mixing ethylene carbonate (EC), propylene carbonate (PC), ethyl propionate (EP), and propyl propionate (PP) sequentially in a volume ratio of 10:15:30:45 to prepare a non-aqueous organic solvent and then, dissolving 1.3 M of LiPF₆ of a lithium salt therein.

An electrolyte solution according to Example 1 was prepared by adding 0.2 wt % of the first compound represented by Chemical Formula 1-1, 1 wt % of the second compound represented by Chemical Formula 2-3, and 0.2 wt % of AgNO₃ to the basic electrolyte solution.

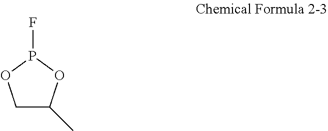

### 2. Manufacture of Rechargeable Lithium Battery Cell

LiCoO₂ as a positive electrode active material, polyvinylidene fluoride as a binder, and ketjen black as a conductive material were mixed in a weight ratio of 97:2:1 and then, dispersed in N-methyl pyrrolidone, preparing positive electrode active material slurry. The positive electrode active material slurry was coated on a 14 μm-thick Al foil current collector and then, dried at 110° C. and compressed, manufacturing a positive electrode.

Negative electrode active material slurry was prepared by mixing artificial graphite as a negative electrode active material, styrene-butadiene rubber as a binder, and carboxylmethyl cellulose as a thickener in a weight ratio of 97:1:2 and then, dispersing the mixture in distilled water. The negative electrode active material slurry was coated on a 10 μm-thick Cu foil current collector and then, dried at 100° C. and compressed, manufacturing a negative electrode.

Subsequently, a 25 μm-thick separator with a polyethylene-polypropylene multi-layer structure was interposed between the positive and negative electrodes to obtain an electrode assembly, the electrode assembly was housed into a pouch-type or kind battery case, and the prepared electrolyte solution was injected thereinto, manufacturing a rechargeable lithium battery cell.

### Examples 2 and 6 and Comparative Examples 1 to 4

Electrolyte solutions and rechargeable lithium battery cells were each manufactured in substantially the same manner as in Example 1 except that the contents of the first compound, the second compound, the Ag salt, and the third compound represented by Chemical Formula 3-1 were changed as shown in Table 1.

TABLE 1

| | First | Second | | Third |
|---|---|---|---|---|
| | compound | compound | Ag salt | compound |
| | (wt %) | (wt %) | (wt %) | (wt %) |
| Example 1 | 0.2 | 1 | 0.2 | 0 |
| Example 2 | 0.5 | 2 | 0.5 | 0 |
| Example 3 | 0.5 | 5 | 1 | 0 |
| Example 4 | 0.2 | 1 | 0.2 | 0.5 |
| Example 5 | 0.5 | 2 | 0.5 | 0.5 |
| Example 6 | 0.5 | 5 | 1 | 0.5 |
| Comparative Example 1 | 0 | 0 | 0 | 0 |
| Comparative Example 2 | 0.5 | 1 | 0 | 0 |
| Comparative Example 3 | 0 | 1 | 0.5 | 0 |
| Comparative Example 4 | 0.5 | 0 | 0.5 | 0 |

### Evaluation Example 1: Evaluation of Battery Performance

The rechargeable lithium battery cells according to Examples 1 to 6 and Comparative Examples 1 to 4 were each charged up to an upper limit of 4.5 V (vs. Li/Li⁺) from 3.0 V under a constant current condition of 0.2 C, paused for 10 minutes, and discharged to 3.0 V under a condition of 0.2 C at 25° C. to perform initial charge and discharge. Herein, initial discharge capacity and initial DC resistance (DC-IR) of the battery cells were each measured and then, provided in Table 2.

Subsequently, the charge and discharge were 200 times repeated at 1 C within a range of 3.0 V to 4.35 V at 25° C. and then, measured with respect to discharge capacity and DC-IR, and the results are shown in Table 2.

A ratio of discharge capacity at the 200th cycle to the initial discharge capacity was calculated and then, provided as capacity retention rate in Table 2. In some embodiments, a ratio of DC-IR at the 200th cycle to the initial DC-IR was calculated and then, shown as a resistance increase rate in Table 2.

TABLE 2

| | Initial | | 200th cycle @25° C. | | Capacity | Resistance |
|---|---|---|---|---|---|---|
| | Discharge | | Discharge | | retention | increase |
| | capacity | DC-IR | capacity | DC-IR | rate | rate |
| | (mAh) | (mΩ) | (mAh) | (mΩ) | (%) | (%) |
| Example 1 | 4572 | 31.1 | 4115 | 36.8 | 90 | 18 |
| Example 2 | 4572 | 32.0 | 4302 | 37.8 | 94 | 18 |
| Example 3 | 4573 | 35.4 | 4038 | 43.3 | 88 | 22 |
| Example 4 | 4574 | 31.5 | 4272 | 35.3 | 93 | 12 |
| Example 5 | 4572 | 33.1 | 4412 | 36.8 | 97 | 11 |
| Example 6 | 4571 | 33.9 | 4219 | 39.2 | 92 | 16 |
| Comparative Example 1 | 4568 | 32.0 | 3746 | 41.7 | 82 | 30 |
| Comparative Example 2 | 4569 | 32.4 | 3897 | 39.3 | 85 | 21 |
| Comparative Example 3 | 4570 | 32.4 | 3866 | 39.7 | 85 | 23 |
| Comparative Example 4 | 4575 | 30.6 | 3811 | 38.3 | 83 | 25 |

Referring to Table 2, each of the examples exhibits improved capacity retention rates and reduced resistance increase rates, compared with the comparative examples not including at least one selected from the first compound, the second compound, and the Ag salt.

In some embodiments, referring to Examples 4 to 6 in which the third compound is added to the three additives, each exhibits much improved capacity retention rates and much reduced resistance increase rates, compared with Examples 1 to 3 in which the third compound is not added to the three additives.

Accordingly, the electrolyte solution prepared by three additives according to one or more embodiments may be applied to improve cycle-life characteristics and concurrently (e.g., simultaneously), effectively suppress or reduce the resistance increase problem, when the rechargeable lithium battery is rapidly charged at a high voltage of 4.5 V or so.

As utilized herein, the terms “substantially,” “about,” and similar terms are used as terms of approximation and not as terms of degree, and are intended to account for the inherent deviations in measured or calculated values that would be recognized by those of ordinary skill in the art.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

While the present disclosure has been described in connection with what is presently considered to be practical example embodiments, it is to be understood that the present disclosure is not limited to the disclosed embodiments. In contrast, it is intended to cover one or more suitable modifications and equivalent arrangements included within the spirit and scope of the appended claims and equivalents thereof.

### REFERENCE NUMERALS

100: rechargeable lithium battery 112: negative electrode
113: separator 114: positive electrode
120: battery case 140: sealing member

What is claimed is:

1. An electrolyte solution for a rechargeable lithium battery, the electrolyte solution comprising:
a non-aqueous organic solvent;
a lithium salt; and
an additive,
wherein the additive comprises:
a first compound that is CsPF₆, a compound represented by Chemical Formula 1, or a combination thereof;
a second compound represented by Chemical Formula 2; and
a silver salt: 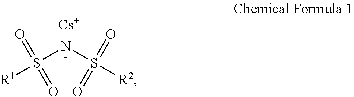
wherein, in Chemical Formula 1, R¹ and R² are each independently a fluoro group or a C1 to C4 fluoroalkyl group substituted with at least one fluoro group, 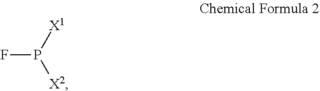
wherein, in Chemical Formula 2, X¹ and X² are each independently a halogen or —O-L¹-R³,
provided that at least one selected from X¹ and X² is —O-L¹-R³, and
wherein L¹ is a single bond, or a substituted or unsubstituted C1 to C10 alkylene group,
R³ is a cyano group, a difluorophosphite group, a substituted or unsubstituted C1 to C10 alkyl group, a substituted or unsubstituted C2 to C10 alkenyl group, a substituted or unsubstituted C3 to C10 cycloalkyl group, a substituted or unsubstituted C3 to C10 cycloalkenyl group, a substituted or unsubstituted C2 to C10 alkynyl group, a substituted or unsubstituted C3 to C10 cycloalkynyl group, or a substituted or unsubstituted C6 to C20 aryl group, and
when both X¹ and X² are —O-L¹-R³,
R³ is each independently present, or
two R³s are linked to form a substituted or unsubstituted monocyclic or polycyclic aliphatic ring, or a substituted or unsubstituted monocyclic or polycyclic aromatic ring.

2. The electrolyte solution of claim 1, wherein:
the compound represented by Chemical Formula 1 is represented by Chemical Formula 1-1 or 1-2:

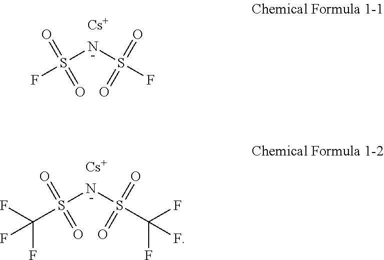

3. The electrolyte solution of claim 1, wherein:
the first compound is in an amount of about 0.05 wt % to about 5.0 wt % based on a total weight of the electrolyte solution.

4. The electrolyte solution of claim 1, wherein:
one selected from X¹ and X² in Chemical Formula 2 is a fluoro group and the other is —O-L²-R⁴, and
wherein L² is a single bond or a substituted or unsubstituted C1 to C10 alkylene group, and
R⁴ is a cyano group or a difluorophosphite group.

5. The electrolyte solution of claim 1, wherein:
the second compound is represented by Chemical Formula 2-1: 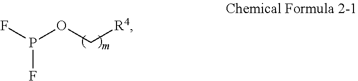
and
wherein, in Chemical Formula 2-1,
m is an integer of 1 to 5, and
R⁴ is a cyano group or a difluorophosphite group.

6. The electrolyte solution of claim 1, wherein:
in Chemical Formula 2,
X¹ is —O-L³-R⁵,
X² is —O-L⁴-R⁶,
L³ and L⁴ are each independently a single bond or a substituted or unsubstituted C1 to C10 alkylene group, and
R⁵ and R& are each independently a substituted or unsubstituted C1 to C10 alkyl group, and R⁵ and Re are linked to form a substituted or unsubstituted monocyclic or polycyclic aliphatic ring.

7. The electrolyte solution of claim 1, wherein:
the second compound is represented by Formula 2-2: 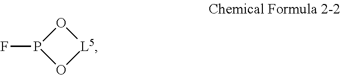
and
wherein, in Chemical Formula 2-2,
L⁵ is a substituted or unsubstituted C2 to C5 alkylene group.

8. The electrolyte solution of claim 1, wherein:
the second compound is represented by Chemical Formula 2-2a or 2-2b: 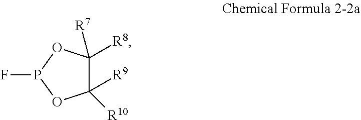
1 wherein, in Chemical Formula 2-2a,
R⁷ to R¹⁰ are each independently hydrogen, a halogen, or a substituted or unsubstituted C1 to C5 alkyl group, 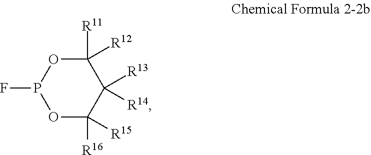
and
wherein, in Chemical Formula 2-2b, R¹¹ to R¹⁶ are each independently hydrogen, a halogen, or a substituted or unsubstituted C1 to C5 alkyl group.

9. The electrolyte solution of claim 1, wherein:
the second compound is any one selected from compounds listed in Group 1:

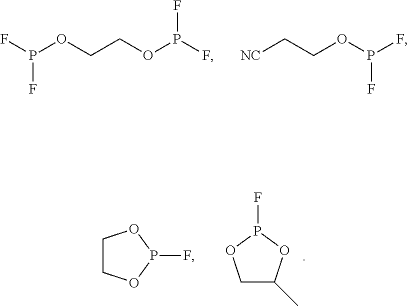

10. The electrolyte solution of claim 1, wherein:
the second compound is in an amount of about 0.1 wt % to about 10 wt % based on a total weight of the electrolyte solution.

11. The electrolyte solution of claim 1, wherein:
the silver salt comprises one or more selected from AgNO₃, AgNO₂, AgN₃, AgCN, AgPF₆, AgN(FSO₂)₂, AgN(CF₃SO₂)₂, AgF, AgSO₃CF₃, and AgBF₄.

12. The electrolyte solution of claim 1, wherein:
the silver salt is in an amount of about 0.1 wt % to about 10 wt % based on a total weight of the electrolyte solution.

13. The electrolyte solution of claim 1, wherein:
the first compound and the second compound are in a weight ratio of about 5:95 to about 40:60.

14. The electrolyte solution of claim 1, wherein:
the first compound and the silver salt are in a weight ratio of about 30:70 to about 70:30.

15. The electrolyte solution of claim 1, wherein:
the silver salt and the second compound are in a weight ratio of about 5:95 to about 40:60.

16. The electrolyte solution of claim 1, wherein:
the additive further comprises a third compound represented by Chemical Formula 3: 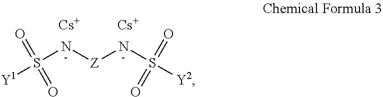
and
wherein, in Chemical Formula 3,
Z is C(═O) or S(═O)₂, and
Y¹ and Y² are each independently a fluoro group or a C1 to C5 fluoroalkyl group substituted with at least one fluoro group.

17. The electrolyte solution of claim 16, wherein:
the third compound is represented by any one selected from Chemical Formulas 3-1 to 3-8: 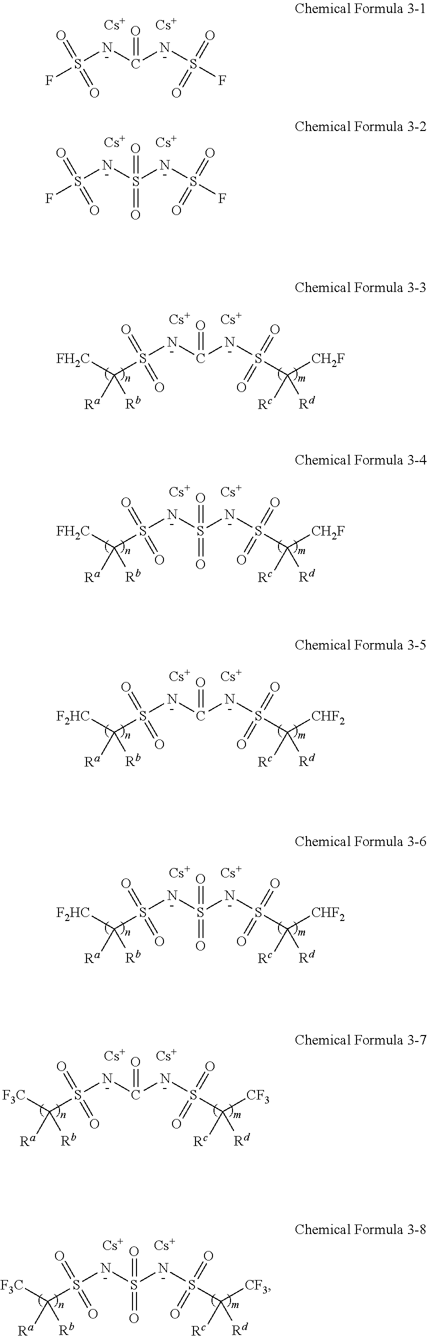
and
wherein, in Chemical Formulas 3-3 to 3-8,
R^{a}, R^{b}, R^{c}, and R^{d} are each independently hydrogen or a fluoro group, and
n and m are each independently an integer of 0 to 4.

18. The electrolyte solution of claim 16, wherein:
the third compound is in an amount of about 0.05 wt % to about 5 wt % based on a total weight of the electrolyte solution.

19. The electrolyte solution of claim 1, wherein:
the electrolyte solution further comprises other additives,
the other additives comprising one or more selected from vinylene carbonate, fluoroethylene carbonate, difluoroethylene carbonate, chloroethylene carbonate, dichloroethylene carbonate, bromoethylene carbonate, dibromoethylene carbonate, nitroethylene carbonate, cyanoethylene carbonate, vinylethylene carbonate, succinonitrile, adiponitrile, 1,3,6-hexanetricyanide, propene sultone, propane sultone, lithium tetrafluoroborate, lithium difluorophosphate, and 2-fluoro biphenyl.

20. A rechargeable lithium battery, comprising
a positive electrode comprising a positive electrode active material;
a negative electrode comprising a negative electrode active material;
a separator between the positive electrode and the negative electrode; and
the electrolyte solution of claim 1.
